(19) United States
(12) Patent Application Publication
(10) Pub. No.: US 2019/0307515 A1
(43) Pub. Date: Oct. 10, 2019

(54) ULTRASOUND DIAGNOSTIC APPARATUS AND PUNCTURE NEEDLE SHIFT ANGLE CALCULATION METHOD

(71) Applicant: Konica Minolta, Inc., Tokyo (JP)

(72) Inventors: Tatsuya NAITO, Tokyo (JP); Morio Nishigaki, Fujisawa-shi (JP); Toshiharu Sato, Tokyo (JP); Takashi Mizuno, Tokyo (JP)

(21) Appl. No.: 16/352,407

(22) Filed: Mar. 13, 2019

(30) Foreign Application Priority Data
Apr. 10, 2018 (JP) 2018-075150

(51) Int. Cl. A61B 34/20; A61B 17/34; A61B 8/08; A61B 8/14; A61B 8/00

(52) U.S. Cl. CPC A61B 34/20; A61B 17/3403; A61B 8/0841; A61B 8/145; A61B 8/4494; A61B 2090/378; A61B 8/5223; A61B 2017/3413; A61B 2034/2063; A61B 2034/2065; A61B 8/5207

(57) ABSTRACT

An ultrasound diagnostic apparatus includes a transmitter which transmits, to an ultrasound probe including a plurality of transducers which are arranged in a plurality of regions in a short axis direction and arranged in a long axis direction in each of the regions and can transmit and receive ultrasound independently in the plurality of regions in the short axis direction, a driving signal in each of the regions, a receiver which receives a receiving signal in each of the regions from the ultrasound probe, and a hardware processor which generates ultrasound image data from the received receiving signal in the region, extracts an image of a puncture needle inserted into a subject from the generated ultrasound image data in the region, and calculates a shift angle of the puncture needle to the ultrasound probe using boundary position information of the extracted image of the puncture needle in the region.

### BACKGROUND

### Technological Field

The present invention relates to an ultrasound diagnostic apparatus and a puncture needle shift angle calculation method.

### Description of the Related art

Conventionally, an ultrasound diagnostic apparatus has been known which irradiates ultrasound into a subject, receives its reflected ultrasound, and performs predetermined signal data processing to generate an ultrasound image of an internal structure of the subject. The ultrasound diagnostic apparatus has been widely used for various applications such as inspection for medical purposes, medical treatment, and inspection of the inside of architectural construction.

The ultrasound diagnostic apparatus has been used to not only display an ultrasound image but also insert, in collecting a sample of a specific site (target) within a subject, discharging water or the like, or injecting or placing a medical agent, a marker, or the like into the specific site, a puncture needle used for the injection and the placement into a position of the target while visually recognizing the puncture needle and the position of the target. Treatment for the target within the subject can be quickly, reliably, and easily performed by using the ultrasound image.

As the ultrasound diagnostic apparatus, an ultrasound diagnostic apparatus, in which transducers which transmit and receive ultrasound are arranged in a one-dimensional or two-dimensional matrix shape in a ultrasound probe and which performs image pickup while subjecting respective positions and directions of the transducers which transmit and receive ultrasound to scanning (electronic scanning in particular) in a predetermined arrangement direction, has been frequently used. The puncture needle is positioned in a range in which image pickup can be consecutively performed in a time period elapsed until the puncture needle reaches a target after being inserted into a subject such as a living body of a patient when inserted into the subject depending on an insertion method by an operation of an operator such as a doctor. Examples of the insertion method include a parallel method as a method for inserting the puncture needle into a target site parallel to a scanning plane (a plane to which a ultrasound beam is emitted) in a direction of the scanning (a lateral direction and a long axis direction) and a crossing method for inserting the puncture needle into a target site by causing the puncture needle to cross the scanning plane. Although the puncture needle has been previously attached to an attachment fixedly connected to an ultrasound probe called a puncturing guide and inserted, the operator may frequently insert the puncture needle freehand at present.

Therefore, the puncture needle may not necessarily be accurately oriented in a desired insertion direction or may be bent depending on an internal state and a structure of the subject and a shape of a distal end of the puncture needle, for example. As a result, a case has occurred where the distal end of the puncture needle deviates from a range in which image pickup can be performed in an elevation direction (a short axis direction) perpendicular to the lateral direction so that image pickup is not performed. Even when a cross-sectional image is simply obtained without using puncture, if an operator is unaccustomed, appropriate change cannot be performed even when an image pickup range in the elevation direction is finely adjusted by changing a posture of the ultrasound probe. Thus, it may take a time and a labor to obtain a desired image.

Therefore, an ultrasound diagnostic apparatus has been known which includes an ultrasound probe in which a plurality of transducers are two-dimensionally arranged in a short axis direction and a long axis direction, and transmits ultrasound beams in two directions, i.e., an ultrasound beam having a deflection angle of zero from all the transducers in the short axis direction and an ultrasound beam having a deflection angle from the transducer at an end in the short axis direction, compares respective intensity characteristics of reflected ultrasound for the deflection angles in the short axis direction in the ultrasound beams in the two directions, specifies an intersection between characteristics respectively obtained by decreasing the intensity characteristics by a difference value between their respective peaks as a short axis position (deflection angle) of the puncture needle, and generates and displays a planar view image of the puncture needle, at a short axis position connecting with the long axis direction, viewed from the top (see Japanese Patent Laid-Open No. 2017-148407).

An ultrasound diagnostic apparatus has been known which includes a two-dimensional array probe (ultrasound probe) in which a plurality of transducers are two-dimensionally arranged and calculates a three-dimensional distal end position of the puncture needle from the time when a transmission signal reaches the transducers at three positions from a transmitter provided in the puncture needle (see Japanese Patent Laid-Open No. 2000-185041). The ultrasound diagnostic apparatus in Japanese Patent Laid-Open No. 2000-185041 calculates the three-dimensional distal end position of the puncture probe by processing for tracking a distal end of the puncture needle using an intensity of reflected ultrasound from the puncture needle, a power intensity of a doppler signal from the puncture needle to which an exciter is attached, and a mutual correlation method in addition to the above.

When the shift angle of the puncture needle is calculated, the method in Japanese Patent Laid-Open No. 2000-185041 presupposes three-dimensional scanning so that a cost for implementation is high. Therefore, a method in Japanese Patent Laid-Open No. 2017-148407 is considered, but presupposes that an intensity of reflection signal depends on only directivity of a short axis beam. However, in an actual subject, an error in the intensity of the reflection signal increases if a site which greatly attenuates partially exists, for example.

### SUMMARY

The present invention is directed to calculating a shift angle of a puncture needle to an ultrasound probe quantitatively, easily, at low cost, and with high accuracy.

To achieve at least one of the abovementioned objects, according to a first aspect of the present invention, an ultrasound diagnostic apparatus reflecting one aspect of the present invention includes:

a transmitter which transmits, to an ultrasound probe including a plurality of transducers which are arranged in a plurality of regions in a short axis direction and arranged in a long axis direction in each of the regions and can transmit and receive ultrasound independently in the plurality of regions in the short axis direction, a driving signal in each of the regions;

a receiver which receives a receiving signal in each of the regions from the ultrasound probe; and

a hardware processor which generates ultrasound image data from the received receiving signal in the region, extracts an image of a puncture needle inserted into a subject from the generated ultrasound image data in the region, and calculates a shift angle of the puncture needle to the ultrasound probe using boundary position information of the extracted image of the puncture needle in the region.

According to a second aspect of the present invention, a puncture needle shift angle calculation method reflecting one aspect of the present invention comprises

transmitting, to an ultrasound probe including a plurality of transducers which are arranged in the plurality of regions in a short axis direction and are arranged in a long axis direction in each of the regions and can transmit and receive ultrasound independently in the plurality of regions in the short axis direction, a driving signal in each of the regions;

receiving a receiving signal in each of the regions from the ultrasound probe;

generating ultrasound image data from the received receiving signal in the region;

extracting an image of a puncture needle inserted into a subject from the generated ultrasound image data in the region; and

calculating a shift angle of the puncture needle to the ultrasound probe using boundary position information of the extracted image of the puncture needle in the region.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an overall view of an ultrasound diagnostic apparatus according to an embodiment of the present invention;

FIG. 2 is a block diagram illustrating an internal configuration of the ultrasound diagnostic apparatus;

FIG. 3 is a diagram illustrating an example of an arrangement of transducers in an ultrasound probe;

FIG. 4A is a schematic side view illustrating a parallel method in ultrasonically guided puncture;

FIG. 4B is a schematic top view illustrating a parallel method in ultrasonically guided puncture;

FIG. 5A is a schematic side view illustrating a crossing method in ultrasonically guided puncture;

FIG. 5B is a schematic top view illustrating a crossing method in ultrasonically guided puncture;

FIG. 6 is a diagram illustrating a schematic configuration in a short axis direction of the ultrasound probe;

FIG. 7 is a flowchart illustrating puncture needle image display processing;

FIG. 8A is a schematic top view of the ultrasound probe, illustrating a shift angle of the puncture needle in the parallel method;

FIG. 8B is a diagram illustrating a composite ultrasound image in the parallel method;

FIG. 8C is a diagram illustrating boundary lines among ultrasound beams;

FIG. 9A is a schematic top view of the ultrasound probe, illustrating the shift angle of the puncture needle in the crossing method;

FIG. 9B is a diagram illustrating a composite ultrasound image in the crossing method;

FIG. 10A is a diagram illustrating a composite display screen;

FIG. 10B is a schematic top view of the ultrasound probe after shift angle adjustment;

FIG. 11 is a schematic top view of the ultrasound probe, illustrating a threshold angle θ_{α};

FIG. 12A is a histogram of a shift angle of a puncture needle; and

FIG. 12B is a diagram illustrating a history information image of the shift angle.

### DETAILED DESCRIPTION OF EMBODIMENTS

Hereinafter, one or more embodiments of the present invention will be described with reference to the drawings. However, the scope of the invention is not limited to the disclosed embodiments. An embodiment and a modification according to the present invention will be described in detail with reference to the accompanying drawings.

### Embodiment

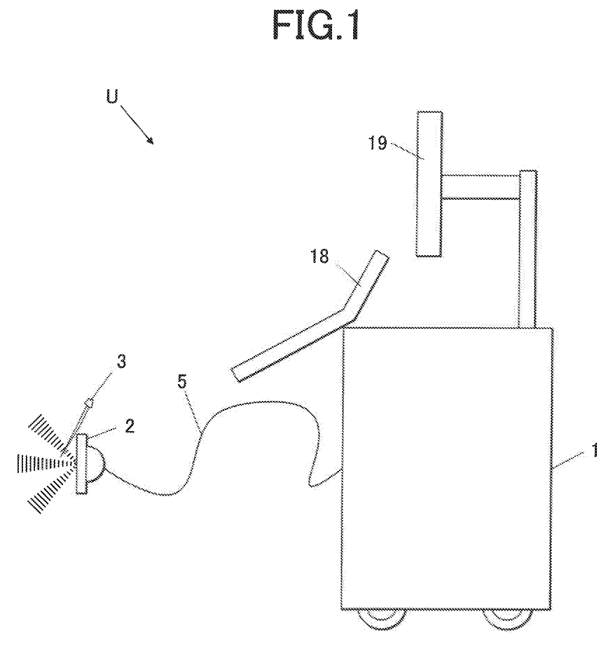

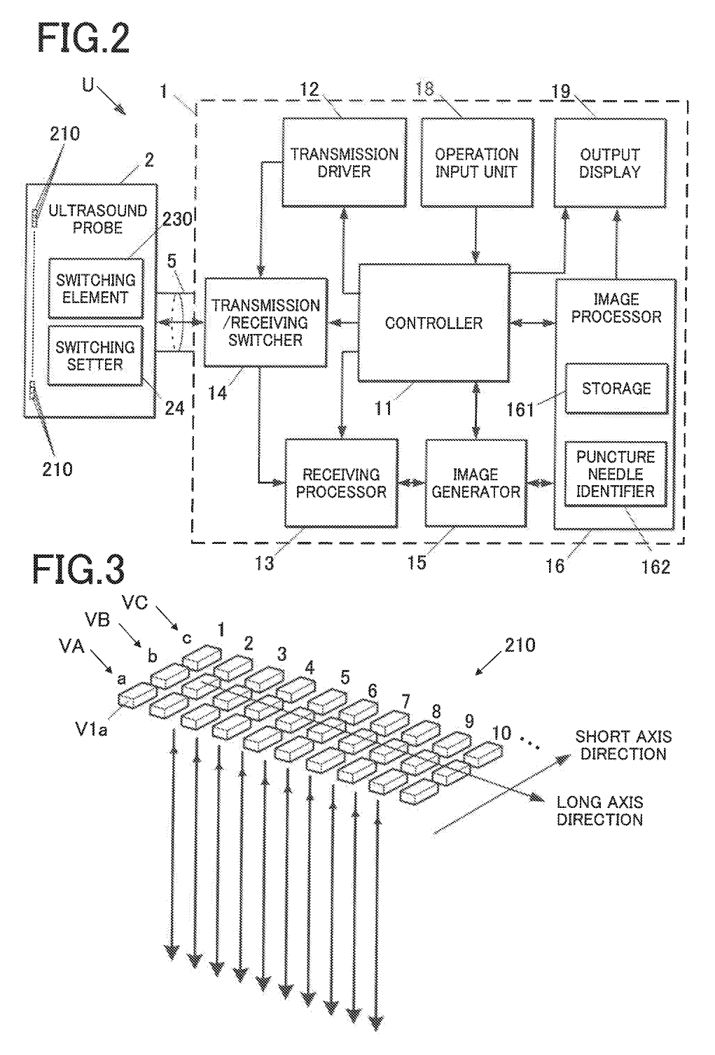

An embodiment of the present invention will be described below with reference to FIGS. 1 to 11. First, an entire apparatus configuration of an ultrasound diagnostic apparatus U according to the present embodiment will be described with reference to FIGS. 1 and 2. FIG. 1 is an overall view of the ultrasound diagnostic apparatus U according to the present embodiment. FIG. 2 is a block diagram illustrating an internal configuration of the ultrasound diagnostic apparatus U.

As illustrated in FIG. 1, the ultrasound diagnostic apparatus U includes an ultrasound diagnostic apparatus body 1, an ultrasound probe 2 connected to the ultrasound diagnostic apparatus body 1 via a cable 5, and a puncture needle 3 which is a treatment instrument as a recognition object.

The puncture needle 3 has a hollow long needle shape, and is inserted into a subject at an angle determined freehand by an operator such as a doctor or an engineer. The puncture needle 3 can be converted into a puncture needle having an appropriate thickness, length, and distal end shape depending on a site (target) to be collected of a subject such as a patient or the type or the amount of a medical agent or the like to be injected. In the ultrasound diagnostic apparatus U, an attachment section as an attachment which guides the puncture needle 3 in a puncture direction and a guide section which is fixedly provided in the ultrasound probe 2 and guides the puncture needle 3 in the puncture direction may be provided.

The ultrasound diagnostic apparatus body 1 is provided with an operation input unit 18 and an output display 19 as a display. As illustrated in FIG. 2, the ultrasound diagnostic apparatus body 1 includes a controller 11 as a hardware processor, a transmission driver 12 as a transmitter, a receiving processor 13 as a receiver, a transmission/receiving switcher 14, an image generator 15, and an image processor 16 as a hardware processor, in addition to the operation input unit 18 and the output display 19. The controller 11 outputs a driving signal to the ultrasound probe 2 to output ultrasound based on an input operation from outside to an input device such as a keyboard or a mouse in the operation input unit 18, acquires a receiving signal relating to ultrasound receiving from the ultrasound probe 2 to perform various types of processing, and displays a result or the like on a display screen or the like of the output display 19, as needed.

The controller 11 includes a CPU (central processing unit), an HDD (hard disk drive), and a RAM (random access memory) as a storage, for example. The CPU reads out various types of programs stored in the HDD and loads the read programs into the RAM, to integrally control respective operations of the units in the ultrasound diagnostic apparatus U according to the programs. The HDD stores a control program and various types of processing programs for causing the ultrasound diagnostic apparatus U to operate and various types of setting data, for example. The HDD particularly stores a puncture needle image display program for performing puncture needle image display processing, described below. The programs and the setting data may be stored such that reading and writing are updatable in an auxiliary storage device using a nonvolatile memory such as a flash memory including an SSD (solid state drive), for example, in addition to the HDD. The RAM is a volatile memory such as an SRAM or a DRAM, and provides a work memory space to the CPU and stores temporary data.

The transmission driver 12 outputs a driving signal to be fed to the ultrasound probe 2 in response to a control signal inputted from the controller 11, and transmits ultrasound to the ultrasound probe 2. The transmission driver 12 includes a clock generation circuit, a pulse width setter, a pulse generation circuit, and a delay circuit, for example. The clock generation circuit is a circuit which generates clock signals to determine a transmission timing and a transmission frequency of a pulse signal. The pulse width setter sets a waveform (shape), a voltage amplitude, and a pulse width of a transmission pulse to be outputted from the pulse generation circuit. The pulse generation circuit generates a transmission pulse as a driving signal based on the setting by the pulse width setter, and outputs the generated transmission pulse to wiring paths which differ for each of transducers 210 in the ultrasound probe 2. The delay circuit counts the clock signals outputted from the clock generation circuit, and causes the pulse generation circuit to generate a transmission pulse and output the generated transmission pulse to each of the wiring paths when a set delay time period elapses.

The receiving processor 13 is a circuit which acquires the receiving signal inputted from the ultrasound probe 2 under the control of the controller 11. The receiving processor 13 includes an amplifier, an A/D (analog to digital) conversion circuit, and a phase-adjustment and addition circuit, for example. The amplifier is a circuit which amplifies receiving signals corresponding to ultrasound received by the transducers 210 in the ultrasound probe 2, respectively, at predetermined amplification factors previously set. The A/D conversion circuit is a circuit which respectively converts the amplified receiving signals into digital data at a predetermined sampling frequency. The phase-adjustment and addition circuit is a circuit which gives the receiving signals, which have been subjected to A/D conversion, delay time periods for wiring paths corresponding to the transducers 210 to adjust their respective time phases and add the time phases to generate sound ray data.

The transmission/receiving switcher 14 performs a switching operation for transmitting a driving signal to the transducers 210 from the transmission driver 12 when emitting (transmitting) ultrasound from the transducers 210 while outputting a receiving signal to the receiving processor 13 when acquiring a signal relating to the ultrasound emitted by the transducers 210 under the control of the controller 11.

The image generator 15 generates a diagnostic image based on ultrasound receiving data. The image generator 15 subjects the sound ray data inputted from the receiving processor 13 to detection (envelope detection) to acquire a signal, and performs logarithmic amplification, filtering (e.g., low-pass transmission or smoothing), and enhancement processing, for example, as needed. The image generator 15 generates as one of diagnostic images frame image data relating to B (Brightness) mode display as a tomographic image representing a two-dimensional structure within a cross section including a transmission direction of a luminance signal corresponding to the intensity of the signal (a depth direction of the subject) and a scanning direction (a lateral direction and a long axis direction in a two-dimensional arrangement of the transducers 210) of the ultrasound transmitted by the ultrasound probe 2. At this time, the image generator 15 can perform dynamic range adjustment and gamma correction relating to display, for example. The image generator 15 can be configured to include a dedicated CPU and RAM used for image generation. Alternatively, in the image generator 15, a dedicated hardware configuration relating to image generation may be formed on a substrate (e.g., an ASIC (application-specific integrated circuit)) or formed by an FPGA (field programmable gate array). Alternatively, the image generator 15 may have a configuration in which the CPU and the RAM in the controller 11 perform processing relating to image generation.

The image processor 16 includes a storage 161 and a puncture needle identifier 162, for example. The storage 161 stores diagnostic image data (frame image data), which is used for real time display or display conforming thereto upon being processed by the image generator 15, corresponding to a predetermined number of latest frames frame by frame. The storage 161 is, for example, a volatile memory such as a DRAM (dynamic random access memory). Alternatively, the storage 161 may be various types of high-speed rewritable nonvolatile memories. The diagnostic image data stored in the storage 161 is read out under the control of the controller 11, is transmitted to the output display 19, and is outputted to outside the ultrasound diagnostic apparatus U via a communicator (not illustrated). At this time, if a display system of the output display 19 is a television system, a DSC (digital scan converter) may be provided between the storage 161 and the output display 19 so that the diagnostic image data is outputted after a scanning format has been converted.

The puncture needle identifier 162 generates image data for identifying a position of the puncture needle 3, performs appropriate processing for the image data to extract and identify a partial needle image at a position including a distal end of the puncture needle 3 and colors the extracted partial needle image of the puncture needle 3. The puncture needle identifier 162 may share a CPU and a RAM in the image processor 16, or may include a dedicated CPU and RAM. Alternatively, the puncture needle identifier 162 may perform various types of processing using the CPU and the RAM in the controller 11. The puncture needle identifier 162 can store and hold distal end position information of the identified puncture needle 3 as a history.

Examples of a method for identifying a position of the puncture needle 3 include a method for finding from ultrasound image data in a plurality of frames a difference and a correlation among the frames to generate movement evaluation information representing evaluation of movement, calculating a movement speed of a distal end of the puncture needle, and detecting a position of the distal end of the puncture needle from the movement speed of the distal end of the puncture needle and the movement evaluation information, to identify a position of the puncture needle including the distal end, as described in Japanese Patent No. 6123458. A method for estimating a subsequent position of a distal end of a puncture needle 3 based on a movement history of the distal end, and detecting the distal end based on the estimated position, to identify a position of the puncture needle including the distal end may be used. A method for an operator selecting one of contour candidates first obtained by performing contour detection using an input operation to the operation input unit 18 and detecting a contour similar to the selected contour candidate, to detect a position of the puncture needle based on the abovementioned estimated position may be used.

The operation input unit 18 includes a push button switch, a keyboard, a mouse, or a trackball or their combinations, and converts a user's input operation into an operation signal and inputs the operation signal to the ultrasound diagnostic apparatus body 1.

The output display 19 includes a display screen using any one of various display systems such as an LCD (liquid crystal display), an organic EL (electro-luminescent) display, an organic EL display, a plasma display, and a CRT (cathode ray tube) display and a driver for the display screen. The output display 19 generates a control signal outputted from the CPU and a driving signal of a display screen (each display pixel) according to the image data generated by the image processor 16, and displays measurement data based on a menu, a status, and a received ultrasound relating to ultrasound diagnosis on the display screen. The output display 19 may be configured to display the presence or absence of turn-on of power by separately including an LED (light emitting diode) lamp, for example.

The operation input unit 18 and the output display 19 may be provided to be integrated with a housing of the ultrasound diagnostic apparatus body 1, or may be attached to the outside via an RGB cable, a USB (universal serial bus) cable, an HDMI (high-definition multimedia interface) cable (registered trademark: HDMI), or the like. If the ultrasound diagnostic apparatus body 1 is provided with an operation input terminal and a display output terminal, the operation input unit 18 and the output display 19 may be used by respectively connecting conventional peripheral devices for operation and for display to the terminals.

The ultrasound probe 2 functions as an acoustic sensor which transmits ultrasound (approximately 1 to 30 MHz) and emits the transmitted ultrasound to a subject such as a living body while receiving a reflection wave (echo) reflected on the subject among the emitted ultrasound and converting the received reflection wave into an electrical signal.

The ultrasound probe 2 includes a plurality of transducers 210 which transmit and receive ultrasound, a plurality of switching elements 230 respectively corresponding to the transducers 210, and a switching setter 24. Although the ultrasound probe 2 is here explained as an ultrasound probe which emits the ultrasound into the subject from the outside (a body surface) and receives its reflection wave, examples of the ultrasound probe 2 include an ultrasound probe having a size and a shape used by being inserted into a digestive tube or a blood vessel or a body cavity, for example. The operator performs ultrasound diagnosis by making a transmission/receiving surface of the ultrasound in the ultrasound probe 2, i.e., a surface in a direction in which the ultrasound is emitted from the transducers 210 contact the subject at predetermined pressure to operate the ultrasound diagnostic apparatus U.

The number of transducers of the transducers 210 is optionally set. Although an electronic scan probe using a linear scanning method is adopted for the ultrasound probe 2 in the present embodiment, either one of an electronic scanning method and a mechanical scanning method may be adopted, or any one of a linear scanning method, a sector scanning method, and a convex scanning method can also be adopted.

The transducers 210 are a plurality of transducers each including a piezoelectric element including a piezoelectric body and electrodes provided at both ends where a charge appears by deformation (expansion and contraction) of the piezoelectric body.

When a voltage pulse as a driving signal is supplied to each of the plurality of the transducers 210, the piezoelectric body in the transducer to which the voltage pulse has been supplied deforms (expands and contracts) in response to an electric field occurring in the piezoelectric body so that ultrasound is transmitted. The transmitted ultrasound is emitted in a position and a direction corresponding to a position and a direction of the transducers 210 included in each of a predetermined number of transducer columns to which the voltage pulse has been supplied, a focusing direction of the transmitted ultrasound, and the magnitude of a shift in timing (a delay). When ultrasound (a reflection wave on the subject) in a predetermined frequency band is incident in one of the transducers 210, the thickness of the piezoelectric body varies (vibrates) with sound pressure of the ultrasound so that a charge corresponding to an amount of the variation occurs. The charge is converted into an electrical signal corresponding to an amount of the charge, and the electrical signal is outputted as a receiving signal.

The switching setter 24 stores a setting of a transmission/receiving sequence of the transducers 210 for performing transmission/receiving of ultrasound in a short axis direction (elevation direction) in the two-dimensional arrangement of the transducers 210, and performs an operation for switching on and off of the switching element 230 corresponding to each of the transducers 210 in response to the setting. The transmission/receiving sequence of the transducers 210 and on/off control of the switching elements 230 will be described below.

The cable 5 includes a connector (not illustrated) to the ultrasound diagnostic apparatus body 1 and a connector (not illustrated) to the ultrasound probe 2, respectively, at both its ends, and the ultrasound probe 2 is configured to be detachably attached to the ultrasound diagnostic apparatus body 1 via the cable 5. The cable 5 may be formed integrally with the ultrasound probe 2.

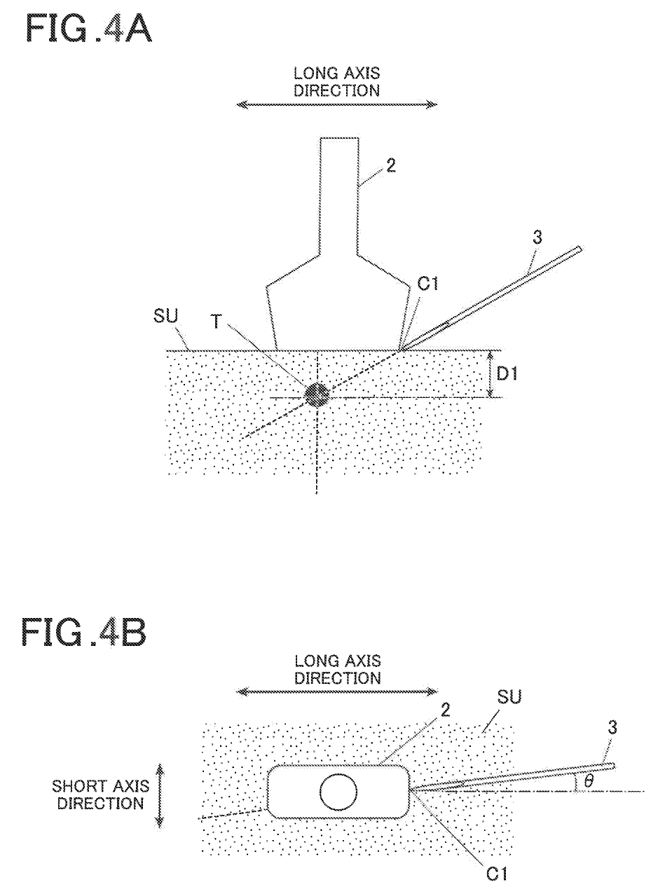

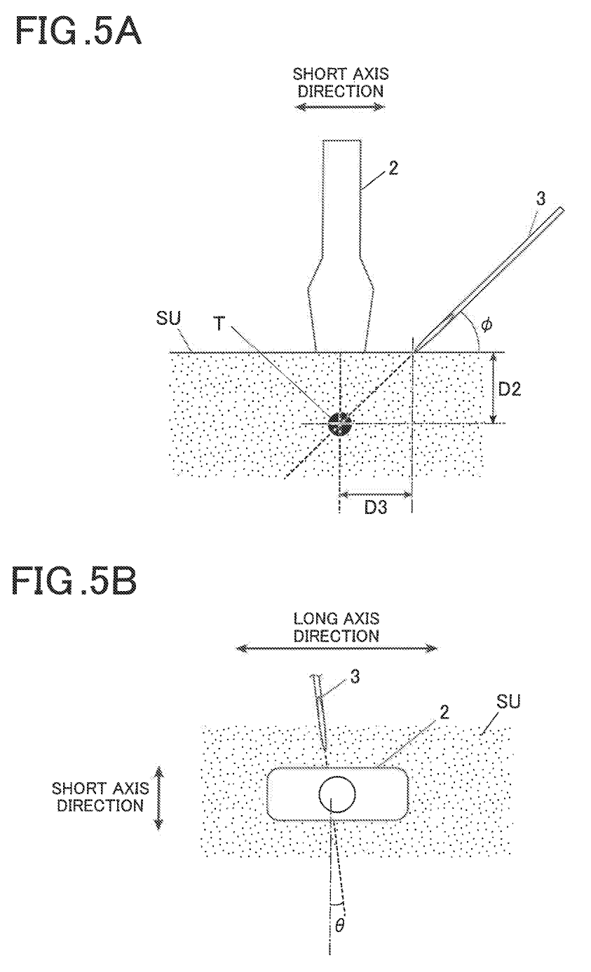

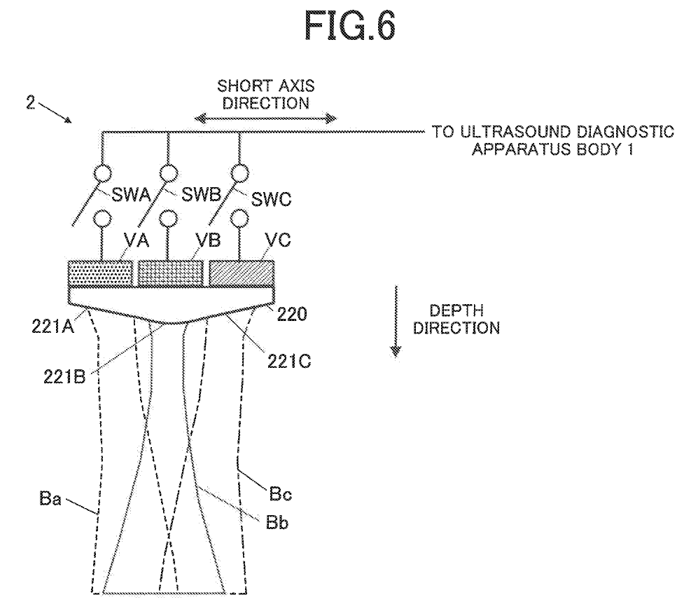

A more detailed configuration and operation of the ultrasound probe 2 will be described with reference to FIGS. 3 to 6. FIG. 3 is a diagram illustrating an example of an arrangement of the transducers 210 in the ultrasound probe 2. FIG. 4A is a schematic side view illustrating a parallel method in ultrasonically guided puncture. FIG. 4B is a schematic top view illustrating a parallel method in ultrasonically guided puncture. FIG. 5A is a schematic side view illustrating a crossing method in ultrasonically guided puncture. FIG. 5B is a schematic top view illustrating a crossing method in ultrasonically guided puncture. FIG. 6 is a diagram illustrating a schematic configuration in a short axis direction of the ultrasound probe 2.

As illustrated in FIG. 3, in the ultrasound diagnostic apparatus U, the transducers 210 are a plurality of transducers arranged in a matrix shape within a two-dimensional plane (which may not be a flat surface) defined by a predetermined lateral direction (scanning direction) and an elevation direction perpendicular to the lateral direction. Generally, the number of arrangements of the transducers 210 in the lateral direction is larger than the number of arrangements of the transducers 210 in the elevation direction, and the lateral direction and the elevation direction are respectively a long axis direction and a short axis direction. The transducers 210 include transducer groups in three columns (columns a, b, and c) in the short axis direction, and transducers in a plurality of stages (stages 1, 2, . . . ) are arranged in the long axis direction in each of the columns. The transducer group in the column a is conveniently represented as transducers VA. Similarly, the transducer groups in the columns b and c are conveniently represented, respectively, as transducers VB and VC. The one transducer in the stage x and the column y is represented as a transducer Vxy.

If a normal B mode (tomographic) image is generated, ultrasound is transmitted and received while the transducers to be driven are sequentially shifted in the long axis direction using the transducers VB in the column b.

A parallel method and a crossing method will be described as a puncture method for the puncture needle 3 in ultrasonically guided puncture with reference to FIGS. 4A to 5B.

As illustrated in FIG. 4A, the parallel method is a method for inserting the puncture needle 3 parallel to a scanning surface in the long axis direction of the ultrasound probe 2 into a target (target side) T, for example, acquiring tissues by puncturing at a depth D1 in a subject SU, as viewed from a cross section (side surface) of the subject SU. As illustrated in FIG. 5A, the crossing method is a method for inserting the puncture needle 3 in a direction crossing the scanning surface in the long axis direction of the ultrasound probe 2 into the target T in the subject SU, as viewed from the cross section (side surface) of the subject SU. The parallel method and the crossing method are differently used depending on uses. Although it may be determined which of the methods is to be used depending on a site to be punctured and a purpose of puncture, the method may be selected by an empirical value of an operator such as a doctor or an engineer.

In the parallel method, if the puncture needle is inserted, the puncture needle 3 is inserted into the subject SU from a long axis end of the ultrasound probe 2, and is inserted toward a depth within a tomographic plane formed by the puncture needle 3 in the long axis direction as the one column corresponding to the transducers VB. If the puncture needle 3 deviates in the short axis direction from the inside of the tomographic surface in this case, the puncture needle 3 is not depicted in a conventional ultrasound diagnostic apparatus.

In the crossing method, the puncture needle 3 is obliquely inserted at an angle ₄ into the subject SU from the short axis side of the ultrasound probe 2, and is inserted into the target T at a depth D2 directly below the ultrasound probe 2 from a position spaced by a distance D3 apart from the target T. When a general ultrasound probe is used in a conventional crossing method, even if a puncture needle 3, which has been inserted into a body surface, reaches a significant depth, the puncture needle 3 is not displayed on an ultrasound image, and an image of the puncture needle first appears in the ultrasound image immediately near a target T. Therefore, it is difficult to know whether the inserted puncture needle 3 advances in a correct direction.

In the present embodiment, the puncture needle 3 is captured in a wide region in both the parallel method and the crossing method. Accordingly, a frame of an ultrasound image by the transducers VA and a frame of an ultrasound image by the transducers VC are obtained in addition to a frame of the ultrasound image by the transducers VB at the same time.

As illustrated in FIG. 4B, in the parallel method, the puncture needle 3 is inserted in the long axis direction and the depth direction from a central portion (central point) C1 in the short axis direction of the long axis end of the ultrasound probe 2. Generally, in the ultrasound probe 2, a mark is formed in the central portion C1 in the short axis direction because the target T is easily aimed at. The operator inserts the puncture needle 3 into a scanning plane passing through the central portion C1 of the ultrasound probe 2 by viewing the ultrasound probe 2 from the top. However, the puncture needle 3 may shift from a reference line in the long axis direction of the scanning plane passing through the central portion C1 of the ultrasound probe 2. An angle of the shift is set to θ.

As illustrated in FIG. 5B, in the crossing method, the puncture needle 3 can also be inserted in the short axis direction and the depth direction from any position in the long axis direction of the ultrasound probe 2. The operator inserts the puncture needle 3 into a surface perpendicular to the scanning plane of the ultrasound probe 2 by viewing the ultrasound probe 2 from the top. However, the puncture needle 3 may shift from a reference line in the short axis direction of a surface perpendicular to the scanning plane of the ultrasound probe 2. An angle of the shift is set to θ.

As illustrated in FIG. 6, the ultrasound probe 2 includes an acoustic lens 220, transducers VA, VB, and VC, and switches SWA, SWB, and SWC in a switching element 230 respectively corresponding to the transducers VA, VB, and VC in the short axis direction viewed from the long axis end. Illustration of an acoustic matching layer arranged between the acoustic lens 220 and the transducers VA, VB, and VC and a backing material arranged on the opposite side to the ultrasound emission direction of the transducers VA, VB, and VC, for example, is omitted.

The acoustic lens 220 is a lens having an aspherical shape which makes ultrasound beams (transmission ultrasound) respectively emitted from the transducers VA, VB, and VC focus. The acoustic lens 220 includes a lens portion 221A through which an ultrasound beam Ba emitted from the transducers VA passes, a lens portion 221B through which an ultrasound beam Bb emitted from the transducers VB passes, and a lens 221C through which an ultrasound beam Bc emitted from the transducers VC passes.

The switch SWA is a switch which can independently turn on and off input of a driving signal to each of the transducers in the transducers VA from the transmission/receiving switcher 14 and output of a receiving signal via the switching setter 24 and the cable 5. The switch SWB is a switch which can independently turn on and off input of a driving signal to each of the transducers in the transducers VB from the transmission/receiving switcher 14 and output of a receiving signal via the switching setter 24 and the cable 5. The switch SWC is a switch which can independently turned on and off input of a driving signal to each of the transducers in the transducers VC from the transmission/receiving switcher 14 and output of a receiving signal via the switching setter 24 and the cable 5.

In the present embodiment, the transducers VA, VB, and VC are arranged such that the ultrasound beams Ba, Bb, and Bc do not substantially overlap one another to a certain depth and a gap is not formed among the ultrasound beams Ba, Bb, and Bc.

A short axis width of the transducers VB has a width wide enough to withstand normal ultrasound scanning. The lens portion 221B in the acoustic lens 220, which covers the transducers VB, has a beam formation capability usable for normal ultrasound scanning. Respective short axis widths of the transducers VA and VC each have a width wide enough to sufficiently obtain a reflection wave (echo) of the inserted puncture needle 3, although the widths may be narrower than that of the transducers VB. Although the lens portions 221A and 221C in the acoustic lens 220, which respectively cover the transducers VA and VC, each desirably have such an aspherical shape that its radius of curvature becomes larger than that of the lens portion 221B, an obliquely flat shape can also be used. Although a shape, which is not oblique but is flat, is not entirely disapproved, the shape may be desirably oblique when fusion with the ultrasound beam Bb by the transducers VB, described below, is considered. A lens shape of the lens portion 221B may be an aspherical shape in an advantage that the lens portion 221B can be smoothly connected to the lens portions 221A and 221C if an inconvenience (e.g., a side robe becomes large) does not arise in normal ultrasound scanning.

To accurately capture a position of the puncture needle 3, positions respectively occupied by the ultrasound beams Ba, Bb, and Bc transmitted and received by the transducers VA, VB, and VC are desirably exclusive. The positions occupied by the ultrasound beams Ba, Bb, and Bc are easily determined by being exclusive because the reflection wave (echo) from the puncture needle 3 is included in only a reflection wave from any one of the transducers VA, VB, and VC.

However, directivity of the ultrasound beam has a shape having a smooth skirt. Thus, the ultrasound beams Ba, Bb, and Bc overlap one another in the skirt. Accordingly, the ultrasound beams Ba, Bb, and Bc are not completely exclusive.

As a peak of the ultrasound beam in the short axis direction, a peak of the ultrasound beam Bb by the transducer VB is desirably used as a reference. However, if the respective short axis widths of the transducers VA and VC are narrower than the short axis width of the transducer VB, for example, it is assumed that respective heights of the ultrasound beams Ba and Bc become lower than a height of the peak of the ultrasound beam Bb. A difference between the heights of the ultrasound beams can be previously found by calculation, and can be corrected using a calculation value. Making the respective heights of peaks of the directivity of the ultrasound beams by the transducers VA and VC equal to the height of the directivity of the ultrasound beam by the transducers VB may be performed. If the transducers greatly differ in sensitivity, a needle position can thus be accurately captured. Since a difference in sensitivity among the transducers occurs depending on a depth, correction may be performed if the difference is large.

An appropriate shape of the acoustic lens 220 in the present embodiment will be described below. First, consider a configuration in which the transducers VA, VB, and VC are covered with an acoustic lens having a shape to which a normal short axis lens is merely connected. In this case, the respective ultrasound beams from the transducers VA, VB, and VC have parallel directivity. However, the ultrasound beam by the transducers VB thins from the vicinity of the transducers VB toward a focus. That is, the ultrasound beams by the transducers VA and VC need to fill the right and the left of the ultrasound beam, which thins, by the transducers VB (have directivity). When in directivity between the ultrasound beam by the transducers VA and the ultrasound beam by the transducers VB, or directivity between the ultrasound beam by the transducers VC and the ultrasound beam by the transducers VB, there is a gap (strictly, a zone where respective sensitivities of both the ultrasound beams are low), the puncture needle 3 becomes difficult to be captured when positioned in the gap. Therefore, the acoustic lens 220 desirably has such a lens shape that the ultrasound beam by the transducers VA and the ultrasound beam by the transducers VC deflect inward.

However, when the respective ultrasound beams by the transducers VA and VC respectively focus at shallow positions close to the transducers VA and VC, the inserted puncture needle 3 does not easily enter the ultrasound beams, i.e., cannot be captured in the puncture using the crossing method. When considered from the foregoing, the respective ultrasound beams by the transducers VA and VC desirably focus at a deep position or does not focus, although they deflect.

If the acoustic lens having such a lens shape that the ultrasound beam by the transducers VA and the ultrasound beam by the transducers VC deflect inward is used, as a depth increases, the respective ultrasound beams by the transducers VA and VC overlap the ultrasound beam at the center by the transducers VB so that the position of the puncture needle 3 cannot be determined. Therefore, the respective ultrasound beams by the transducers VA, VB, and VC are desirably separated from one another such that a deflection angle is not made too large and the position of the puncture needle 3 can be determined up to a depth at which there is no clinical problem.

To which depth the ultrasound beams are separated from one another depends on a diagnostic site. However, the ultrasound beams can be desirably separated from one another up to 25 to 30 [mm] when a high-frequency linear probe is used as the ultrasound probe 2, for example. Examples of a shape of the acoustic lens matching the abovementioned condition include a lens shape of the acoustic lens 220 having the aspherical shape as illustrated in FIG. 6. The acoustic lens 220 has a shape in which a curvature is strong (a radius of curvature is small) in the lens portion 221B corresponding to the transducers VB and a curvature is weak (a radius of curvature is large) in the lens portions 221A and 221C corresponding to the transducers VA and VC.

Although the acoustic lens 220 is used, as illustrated in FIG. 6, in the parallel method, if the switch SWB is turned on and the switches SWA and SWC are turned off, ultrasound is transmitted and received using the transducers VB. However, for an operation in this case, the transmission and receiving of the ultrasound do not differ from transmission and receiving of ultrasound by a conventional ultrasound diagnostic apparatus.

If the switch SWA is turned on and the switches SWB and SWC are turned off, ultrasound is transmitted and received using the transducers VA. However, the lens portion 221A corresponding to the transducers VA has a substantially oblique aspherical shape. Accordingly, a transmission/receiving beam of the ultrasound deflects toward the center of the transducers, and an intersection between the transmission/receiving beam and a center line is at a position farther than a focusing point of the lens portion 221B corresponding to the transducers VB. If the lens portions 221A and 221C are respectively provided with curvatures, the curvatures are each desirably such a curvature that the transmission/receiving beam focuses in the vicinity of the intersection. The transmission/receiving beams of the ultrasound respectively formed by the acoustic lens 220 and the transducers VA, VB, and VC do not overlap one another to a desired depth, and a gap (a dead angle on sensing) is not formed among the transmission/receiving beams. In the present embodiment, not only a tomographic image using the transducers VB but also tomographic images respectively using the transducers VA and VC are formed in the parallel method. Accordingly, a puncture needle, which has deviated from a surface of the tomographic image using the transducers VB, can be captured.

For the crossing method, in the ultrasound probe 2 according to the present embodiment, the target T in the subject SU is within the tomographic image using the transducers VB, as illustrated in FIG. 5A. On the other hand, the inserted puncture needle 3 can be captured much faster than when a normal ultrasound probe is used in the tomographic image using the transducers VA (or the transducers VC). Thus, if the crossing method according to the present embodiment is performed, a position of the puncture needle 3 can be confirmed significantly before the target T, and puncture work can be made easy.

A transmission/receiving sequence of the transducers 210 according to the present invention will be described below with reference to FIG. 3. As described above, in a configuration using the acoustic lens 220, the transducers VA, VB, and VC, and the switches SWA, SWB, and SWC, ultrasound is transmitted and received using the transducers VA and VB to obtain a reflection wave (echo) of the puncture needle 3, which has deviated from the ultrasound beam Bb formed by the transducers VB. However, transducers V1a, V1b, V1c, V2a, V2b, V2c, V3a, V3b, V3c, . . . can be subjected in this order to scanning (ultrasound transmission/receiving) in this case, for example.

The scanning sequence is stored in the switching setter 24.

However, the number of times of transmission/receiving increases to three times in this case. Accordingly, a frame rate for B mode tomographic image display decreases to one-third. Therefore, the transducers V1a, V1b, V1c, V2b, V3a, V3b, V3c, V4b, V5a, V5b, V5c, . . . are scanned in this order, for example, by thinning out the scanning of the transducers VA and VC for capturing the puncture needle 3 so that the frame rate can be inhibited from decreasing. Although a case where one transducer is used for scanning in the long axis direction for simplicity has been described above, a plurality of transducers are actually used to form a transmission/receiving beam in the long axis direction. In addition, an already known method for increasing the frame rate by using parallel receiving in the long axis direction, for example, can also be applied.

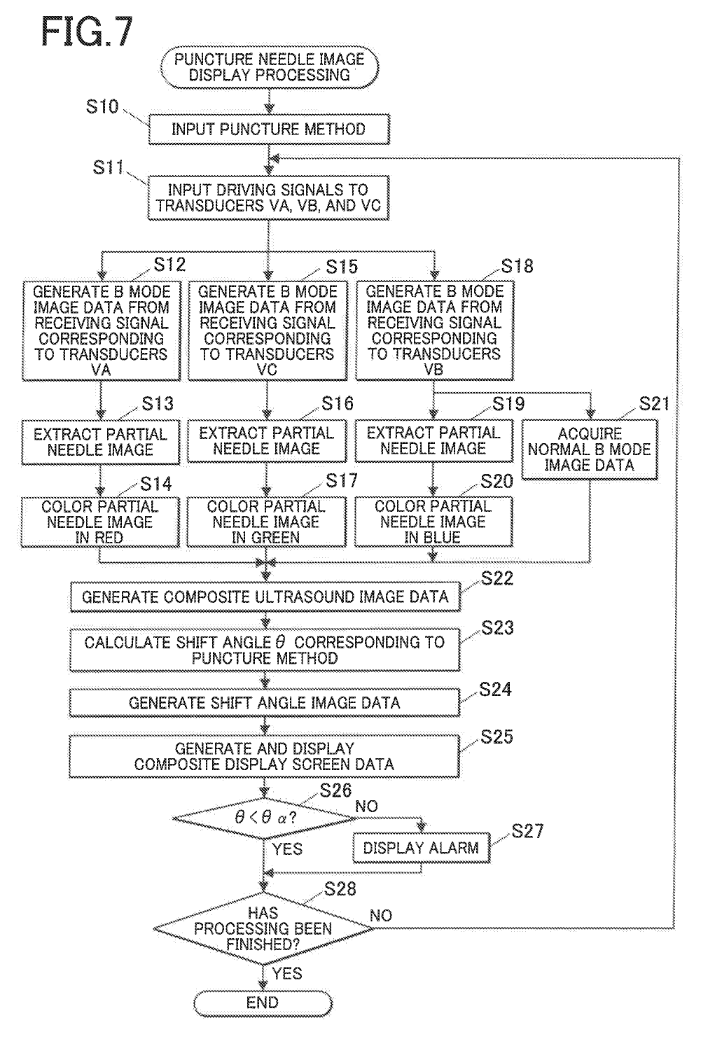

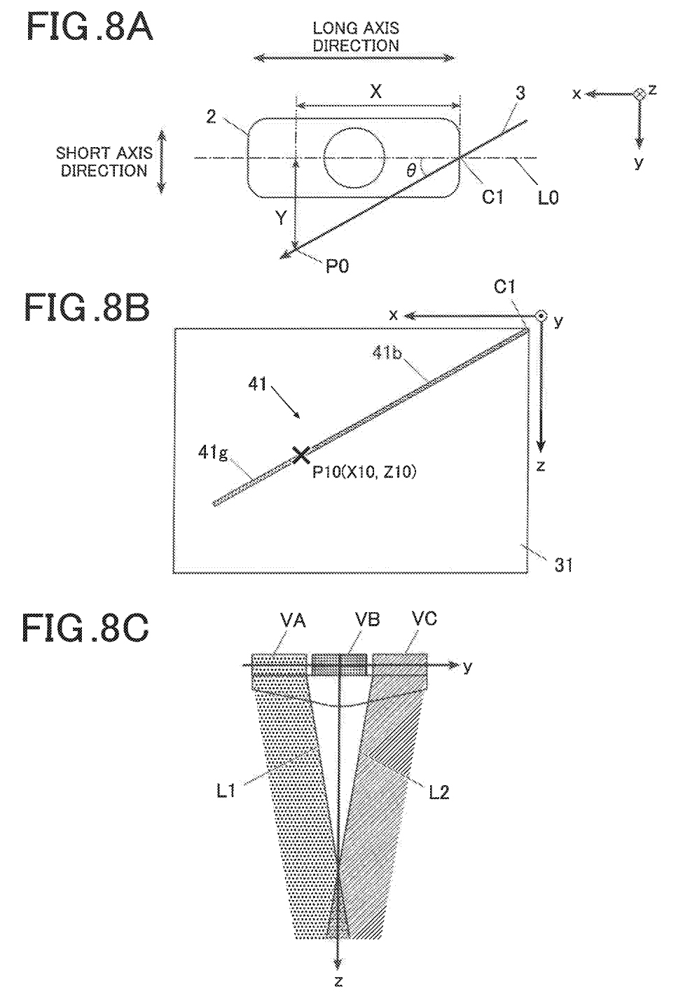

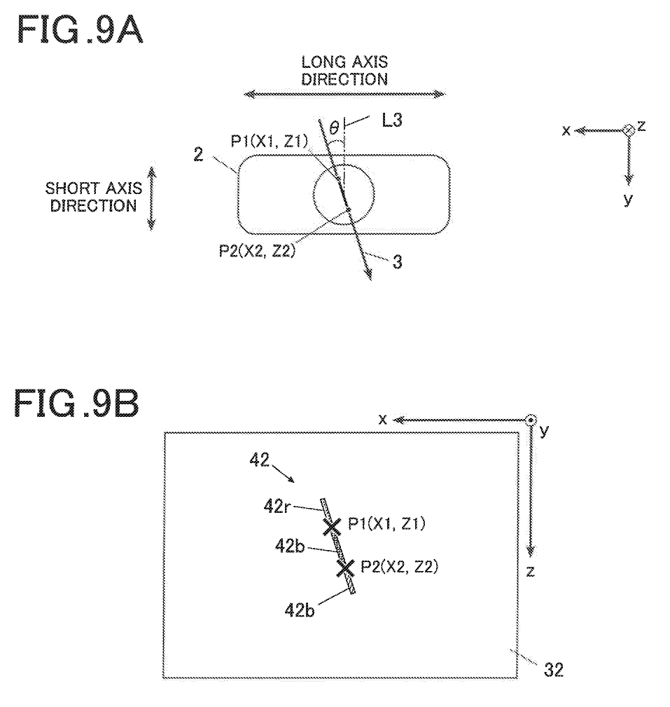

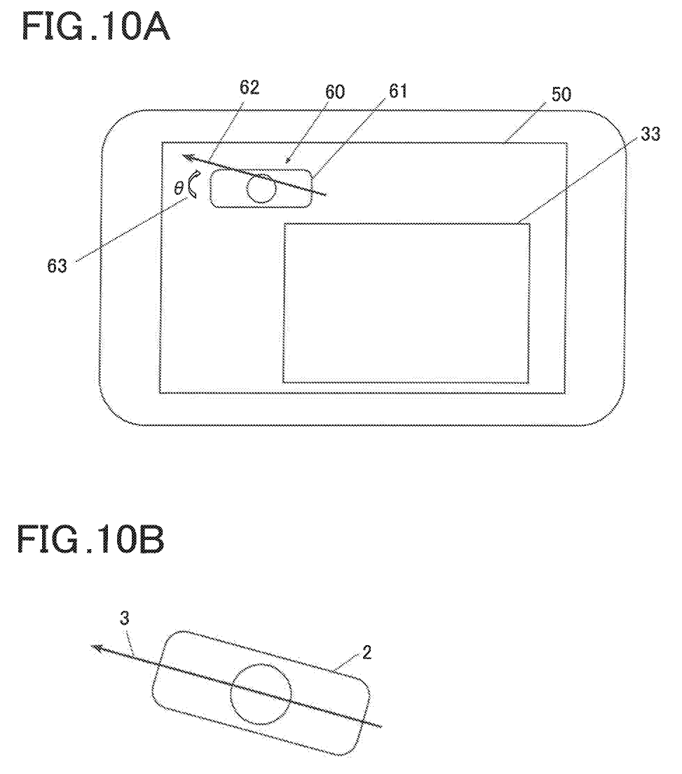

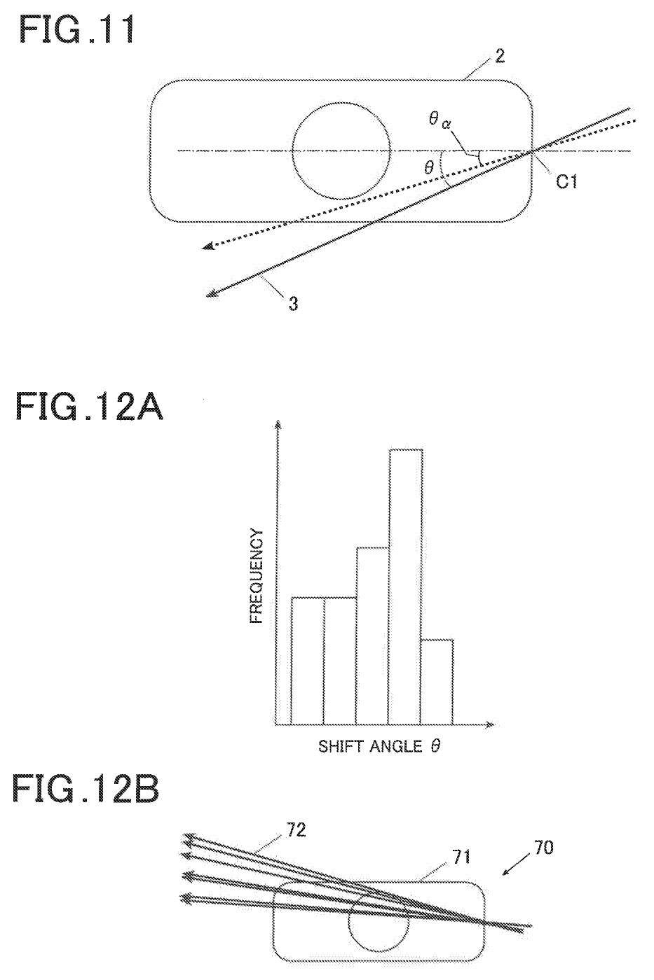

An operation of the ultrasound diagnostic apparatus U will be described below with reference to FIGS. 7 to U. FIG. 7 is a flowchart illustrating puncture needle image display processing. FIG. 8A is a schematic top view of the ultrasound probe 2, illustrating the shift angle θ of the puncture needle 3 in the parallel method. FIG. 8B is a diagram illustrating a composite ultrasound image 31 in the parallel method. FIG. 8C is a diagram illustrating boundary lines L1 and L2 among the ultrasound beams Ba, Bb, and Bc. FIG. 9A is a schematic top view of the ultrasound probe 2, illustrating the shift angle θ of the puncture needle 3 in the crossing method. FIG. 9B is a diagram illustrating a composite ultrasound image 32 in the crossing method. FIG. 10A is a diagram illustrating a composite display screen 50. FIG. 10B is a schematic top view of the ultrasound probe 2 after shift angle adjustment. FIG. 11 is a schematic top view of the ultrasound probe 2, illustrating a threshold angle θα.

Puncture needle image display processing performed by the ultrasound diagnostic apparatus U will be described with reference to FIG. 7. The puncture needle image display processing is processing for subjecting, when an operator such as a doctor or an engineer performs puncture work for inserting the puncture needle 3 into the target T as an object the tissue of which is to be acquired by puncturing the subject SU, for example, a B mode tomographic image of the puncture needle 3 within the subject to live display to assist in the puncture work.

An operator such as a doctor or an engineer previously waits in a consultation room where the ultrasound diagnostic apparatus U is provided, and a patient as the subject SU enters the consultation room and lies down on a bed, and is ready for puncture work using the puncture needle 3, for example. In the ultrasound diagnostic apparatus U, the controller 11 performs puncture needle image display processing according to a puncture needle image display program stored in the ROM using receiving of respective instructions to input various types of setting information such as a frame rate in the puncture needle image display processing and execute the puncture needle image display processing from the operator via the operation input unit 18 as a trigger.

First, the controller 11 accepts input of the puncture method (the parallel method or the crossing method) from the operator via the operation input unit 18.

The controller 11 starts to cause the transmission driver 12 to generate a driving signal and input the driving signal to each transducer of the transducers VA, VB, and VC by switching the switching element 230 corresponding to the transmission/receiving sequence stored in the switching setter 24 via the transmission/receiving switcher 14 to emit transmission ultrasound and receive reflection ultrasound (echo), and causes the receiving processor 13 to acquire a receiving signal via the transmission/receiving switcher 14 (step S11). As the receiving signal obtained in step S11, receiving signals for each frame at the same time respectively corresponding to the transducers VA, VB, and VC are acquired in order corresponding to the transmission/receiving sequence.

The controller 11 causes the image generator 15 to generate B mode image data in one frame from the receiving signal corresponding to the transducers VA inputted from the receiving processor 13 in step S11 (step S12). The controller 11 causes the puncture needle identifier 162 to extract a partial needle image of the puncture needle 3 from B mode image data corresponding to the transducers VA generated in step S12 (discard a portion other than the partial needle image) (step S13). The controller 11 causes the puncture needle identifier 162 to color the partial needle image in the image data generated in step S13 in a red color representing the transducers VA (step S14).

The controller 11 causes the image generator 15 to generate B mode image data in one frame from the receiving signal corresponding to the transducers VC inputted from the receiving processor 13 in step S11 (step S15). The B mode image data generated in step S15 becomes a frame at the same time as the B mode image data generated in step S12. The controller 11 causes the puncture needle identifier 162 to extract a partial needle image of the puncture needle 3 from the B mode image data corresponding to the transducers VC generated in step S15 (step S16). The controller 11 causes the puncture needle identifier 162 to color the partial needle image in the image data generated in step S16 in a green color representing the transducers VC (step S17).

The controller 11 causes the image generator 15 to generate B mode image data in one frame from the receiving signal corresponding to the transducers VB inputted from the receiving processor 13 in step S11 (step S18). The B mode image data generated in step S18 becomes a frame at the same time as the B mode image data respectively generated in steps S12 and S15. The controller n causes the puncture needle identifier 162 to extract a partial needle image of the puncture needle 3 from the B mode image data corresponding to the transducers VB generated in step S18 (step S19). The controller 11 causes the puncture needle identifier 162 to color the partial needle image in the image data generated in step S19 in a blue color representing the transducers VB (step S20).

In steps S14, S17, and S20, which of the transducers VA, VB, and VC has obtained the partial needle image is determined by making display colors as the type of representation different. A combination of the display colors in steps S14, S17, and S20 is one example, and is not limited to this. For example, a gradation like green-blue-violet may be used. Further, the type of representation which can be identified for each of the partial needle images may be changed to another type. Examples of the representation which can be identified for each of the partial needle images may include a configuration in which the partial needle images are made different in saturation and luminance, a configuration in which the partial needle images are made different in presence or absence of flashing, gap, and the like, or a configuration obtained by combining a plurality of types of representations.

In steps S11, S12, S15, and S18, processing corresponding to each of the types of setting information first inputted is performed. A configuration in which various types of setting information are changed and inputted, as needed, from the operator via the operation input unit 18 during execution of the puncture needle image display processing may be used. A configuration in which the respective representations (display colors) of the partial needle images in steps S14, S17, and S20 are inputted as various types of setting information from the operator via the operation input unit 18 may be used.

After step S18 is executed, the controller n causes the image generator 15 to acquire the normal B mode image data in one frame generated in step S11 (step S21). The controller 11 causes the image processor 16 to synthesize the partial needle image in red generated in step S14, the partial needle image in green generated in step S17, the partial needle image in blue generated in step S20, and the B mode image data in one frame acquired in step S21, to generate composite ultrasound image data in one frame (step S22).

The controller 11 calculates the shift angle θ of the puncture needle 3 to correspond to the puncture method inputted in step S10 from the composite ultrasound image data generated in step S22 or each of the image data generated in steps S14, S17, and S20 (step S23). If boundary positions on boundary surfaces among the respective partial needle images in the colors can be calculated, as described below, the shift angle θ can be found. The composite ultrasound image data generated in step S22 includes the partial needle images in all the colors, and the boundary positions among the partial needle images in the colors can be acquired. The boundary positions among the partial needle images in the colors can also be acquired from each of the respective image data including the partial needle images in the colors before the synthesis generated in steps S14, S17, and S20. When the shift angle θ is calculated from each of the image data generated in steps S14, S17, and S20, step S23 may be moved to be executed immediately before step S22.

A method for calculating the shift angle θ of the puncture needle 3 corresponding to the parallel method will be described with reference to FIGS. 8A to 8C. As illustrated in FIG. 8A, the puncture needle 3 is inserted into the central portion C1 in the short axis direction of the long axis end in the ultrasound probe 2 in the parallel method. An x-axis is defined in the long axis direction of the ultrasound probe 2, a y-axis is defined in the short axis direction of the ultrasound probe 2, and a z-axis is defined in a depth direction perpendicular to the x-axis and the y-axis.

A shift angle θ between a reference line LO in an x-axis direction passing through the central portion C1 and the puncture needle 3 can be calculated using a length X in the x-axis direction from the central portion C1 to any point PO on the puncture needle 3 and a length Y in a y-axis direction from the central portion C1.

As an example, a method for calculating the shift angle θ of the puncture needle 3 corresponding to the parallel method when the composite ultrasound image data in the parallel method generated in step S22 represents a composite ultrasound image 31 illustrated in FIG. 8B will be described. The composite ultrasound image 31 includes a needle image 41 of the puncture needle 3 inserted from the central portion C1. The needle image 41 includes a blue partial needle image 41b and a green partial needle image 41g. In the composite ultrasound image 31 illustrated in FIG. 8B, the blue color is represented by a “lattice pattern”, and the green color is represented by “hatching. The same is true for FIG. 9B. A boundary point between the blue partial needle image 41b and the green partial needle image 41g is set to a boundary point P10 (X10, Z10).

To find a value of Y (equal to Y10) corresponding to the boundary point P10 (X10, Z10), boundary lines L1 and L2 for identifying colors illustrated in FIG. 8C are used. The boundary line L1 is simply expressed by y=az−b, and is set as a straight line on a boundary surface between an ultrasound beam corresponding to the red color by the transducer VA and an ultrasound beam corresponding to the blue color by the transducer VB. The boundary line L2 is simply expressed by y=−az+b, and is set as a straight line on a boundary surface between the ultrasound beam corresponding to the blue color by the transducer VB and an ultrasound beam corresponding to the green color by the transducer VC. At this time, the central portion C1 in the short axis direction is set as an origin.

In this case, a slope a generally affects a curvature of an acoustic lens, for example. An intercept b depends on a division width of a transducer in a short axis direction and a position on a boundary surface for identifying colors, for example. Accordingly, the slope a and the intercept b are previously measured or theoretically calculated to set their respective values. Although simply expressed by linear equations, the boundary lines L1 and L2 may be respectively expressed by more complicated equations, for example, may change depending on a depth.

The controller 11 substitutes X10 and Z10 at the boundary point P10 of a boundary between the blue color and the green color of the composite ultrasound image 31 into the boundary line L2 (y=−az+b) between the blue color and the green color, to calculate a value of y (Y10) and calculate the shift angle θ of the puncture needle 3 by the following equation (1) using X10 and Y10, for example:

θ=tan⁻¹(Y10/X10) (1)

A method for calculating the shift angle θ of the puncture needle 3 corresponding to the crossing method will be described with reference to FIGS. 9A and 9B. As illustrated in FIG. 9A, the puncture needle 3 is inserted from any position in the long axis direction in the ultrasound probe 2 in the crossing method. An x-axis, a y-axis, and a z-axis are defined, like in FIG. 8A.

A shift angle θ between a reference line L3 in the y-axis direction and the puncture needle 3 can be calculated using two boundary points for identifying colors of a needle image of the puncture needle 3 in an ultrasound image. However, the shift angle θ of the puncture needle 3 in the crossing method presupposes that partial needle images of the puncture needle 3 in the ultrasound image are respectively in three colors, a red color, a blue color, and a green color (the puncture needle 3 passes through a boundary surface between the red color and the blue color and a boundary surface between the blue color and the green color).

As an example, a method for calculating the shift angle θ of the puncture needle 3 corresponding to the crossing method when the composite ultrasound image data in the crossing method generated in step S22 represents a composite ultrasound image 32 illustrated in FIG. 9B will be described. The composite ultrasound image 32 includes the needle image 42 of the inserted puncture needle 3. The needle image 42 includes a red partial needle image 42r, a blue partial needle image 42b, and a green partial needle image 42g. In the composite ultrasound image 32 illustrated in FIG. 9B, the red color is represented by a “shading (dot) pattern”. A boundary point between the red partial needle image 42r and the blue partial needle image 42b is set as a boundary point P1 (X1, Z1). A boundary point between the blue partial needle image 42b and the green partial needle image 42g is set as a boundary point P2 (X2, Z2).

To find a value of Y (equal to Y1) corresponding to the boundary point P1 (X1, Z1) and a value of Y (equal to Y2) corresponding to the boundary point P2 (X2, Z2), the boundary lines L1 and L2 for identifying colors illustrated in FIG. 8C are used. For example, a point corresponding to the central portion C1 in the short axis direction is set as an origin, like in the parallel method. Although simply expressed by linear equations, the boundary lines L1 and L2 may be respectively expressed by more complicated equations, for example, may change depending on a depth.

For example, the controller 11 substitutes X1 and Z1 at the boundary point Pb of the boundary between the red color and the blue color of the needle image 42 in the composite ultrasound image 32 into the boundary line L1 (y=az−b) between the red color and the blue color, to calculate a value of y (=Y1), substitutes X2 and Z2 at the boundary point P2 between the blue color and the green color of the needle image 42 into the boundary line L2 (y=−az+b) between the blue color and the green color, to calculate a value of y (=Y2), and calculates the shift angle θ of the puncture needle 3 by the equation (2) using X1, Y1, X2, and Y2:

θ=tan⁻¹(|X1−X2|/|Y1−Y2|) (2)

Although the shift angle θ of the puncture needle 3 is calculated using the method for calculating the shift angle θ of the puncture needle 3 corresponding to the puncture method (the parallel method or the crossing method) inputted in step S10 in step S23, the present invention is not limited to this. In step S23, the controller 11 may be configured to automatically judge the puncture method from the composite ultrasound image data generated in step S22 or each of the image data generated in steps S14, S17, and S20 and calculate the shift angle θ of the puncture needle 3 using the method for calculating the shift angle θ corresponding to the judged puncture method. In the configuration in which the puncture method is automatically judged, step S10 may be omitted.

As a first judgment method for automatically judging the puncture method, the controller n inserts the puncture needle 3 while judging the puncture method in response to respective color information of the partial needle images of the composite ultrasound image data generated in step S22 or each of the image data generated in steps S14, S17, and S20 as a frame including the partial needle images first in time. More specifically, the controller 11 judges the puncture method as the parallel method because the puncture needle 3 is inserted into the central portion C1 when the color information of the partial needle image obtained first in time is the blue color and judges the puncture method as the crossing method because the puncture needle 3 is inserted from the side of a short axis end of the ultrasound probe 2 when the color information is the red color or the green color. The color information of the partial needle image obtained first in time is stored in the RAM or the storage 161 in the controller 11, and is also used in the judgment of the puncture method in subsequent frames, for example. For example, the puncture method is judged as the parallel method because the partial needle image 41b appears first in time in a case of the composite ultrasound image 31 illustrated in FIG. 8B and is judged as the crossing method because the red partial needle image 42r appears first in time in a case of the composite ultrasound image 32 illustrated in FIG. 9B.

As a second judgment method for automatically judging the puncture method, the controller n judges the puncture method as the parallel method when the linear partial needle image extends from a left end or a right end of the ultrasound image from the composite ultrasound image data generated in step S22 or each of the image data generated in steps S14, 17, and S20 and judges the puncture method as the crossing method when the linear partial needle image does not extend from the left end or the right end of the ultrasound image and is positioned in a central portion of the ultrasound image. For example, in the composite ultrasound image 31 illustrated in FIG. 8B, the puncture method is judged as the parallel method because the linear needle image 41 extends from a right end of the composite ultrasound image 31. In the composite ultrasound image 32 illustrated in FIG. 9B, the puncture method is judged as the crossing method because the linear needle image 42 does not extend from a left end or a right end of the composite ultrasound image 32 but is positioned in a central portion of the composite ultrasound image 32.

Referring to FIG. 7 again, after step S23 is executed, the controller 11 generates shift angle image data representing the shift angle θ calculated in step S23 (step S24). An example of the shift angle image data generated in step S24 will be described with reference to FIG. 10A. The shift angle image data includes a shift angle image 60 in a composite display screen 50, described below. The shift angle image 60 includes an ultrasound probe image 61, a puncture needle image 62, and a shift angle 63. The ultrasound probe image 61 is a schematic plan view of the ultrasound probe 2 viewed from the top (from the side of the cable 5). The puncture needle image 62 is a needle image representing an insertion direction (and an insertion position) of the puncture needle 3 viewed from the top corresponding to the ultrasound probe image 61. The puncture needle image 62 is a puncture needle image in the parallel method as an example.

The shift angle 63 is an image including a numerical value of the shift angle θ calculated in step S24 and a rotation arrow for setting the shift angle θ to zero and represents a shift angle of the puncture needle image 62 from a reference line of the ultrasound probe image 61 (a line passing through a central portion in the short axis direction of a long axis end and parallel to the short axis direction in the parallel method, illustration of which is omitted).

Referring to FIG. 7 again, after step S24 is executed, the controller 11 synthesizes the composite ultrasound image data corresponding to one frame generated in step S22 and the shift angle image data generated in step S24, to generate composite display screen data and display the generated composite display screen data on the output display 19 (step S25). In step S25, composite display screen data for displaying the composite display screen 50 illustrated in FIG. 10A is generated, for example. The composite display screen 50 includes a composite ultrasound image 33 of the composite ultrasound image data including the needle image in a maximum of three colors generated in step S22 and the shift angle image 60 in the shift angle image data generated in step S24.

The operator who has referred to the displayed shift angle image 60 can rotate and shift the ultrasound probe 2 in a direction indicated by a rotation arrow viewed from the top to set the shift angle θ to zero degrees with the puncture needle 3 fixed, as illustrated in FIG. 10B. However, the operator who has referred to the displayed shift angle image 60 can also insert the puncture needle 3 again to set the shift angle θ to zero degrees with the ultrasound probe 2 fixed as viewed from the top.

Referring to FIG. 7 again, after step S24 is executed, the controller 11 judges whether or not the shift angle θ of the puncture needle 3 to the reference line calculated in step S23 is less than the threshold angle θ_{α} in the parallel method or the crossing method previously set (step S26). The threshold angle θ_{α} is a threshold angle for judging whether or not the shift angle θ of the puncture needle 3 is large enough to require a warning. Threshold angles are separately prepared, respectively, in the parallel method and the crossing method.

As illustrated in FIG. 11, when the ultrasound probe 2 is viewed from the top in the parallel method, the controller 11 judges whether or not the shift angle θ corresponding to the puncture needle 3 is less than the threshold angle θ_{α}. The threshold angle θ_{α} is stored in a nonvolatile memory such as the HDD in the controller 11, and is read out and used, for example. The threshold angle θ_{α} stored in the nonvolatile memory may be changed, as needed, depending on input by the operator via the operation input unit 18.

If the shift angle θ is the threshold angle θ_{α} or more (YES in step S26), the controller 11 generates alarm information for warning that the shift angle θ of the puncture needle 3 is the threshold angle θ_{α} or more and displays the generated alarm information to the output display 19 (step S27). After step S27 is executed or if the shift angle θ is less than the threshold angle θ_{α} (YES in step S26), the controller 11 judges whether or not puncture needle image display processing is finished depending on the presence or absence of input of an instruction to finish the puncture needle image display processing from the operator via the operation input unit 18 (step S28). If the puncture needle image display processing is not finished (NO in step S28), the processing proceeds to step S11. If the puncture needle image display processing is finished (YES in step S28), the processing ends.

As described above, according to the present embodiment, the ultrasound diagnostic apparatus U includes the plurality of transducers respectively arranged in the plurality of regions (the transducers VA, VB, and VC) in the short axis direction and arranged in the long axis direction in each of the regions, and includes the transmission driver 12 which transmits the driving signal in each of the regions to the ultrasound probe 2 capable of transmitting and receiving ultrasound independently in the plurality of regions in the short axis direction and the receiving processor 13 which receives the receiving signal in each of the regions from the ultrasound probe 2. The ultrasound diagnostic apparatus U includes the image generator 15 which generates the ultrasound image data from the received receiving signal in each of the regions, the image processor 16 which extracts the image of the puncture needle inserted into the subject from the generated ultrasound image data in the region, and the controller 11 which calculates the shift angle θ of the puncture needle 3 to the ultrasound probe 2 using boundary position information of the extracted image of the puncture needle in the region.

Therefore, the shift angle of the puncture needle to the ultrasound probe 2 can be calculated quantitatively, easily, at low cost, and with high accuracy.

The controller 11 displays the shift angle image data representing the calculated shift angle θ of the puncture needle 3 on the output display 19. Accordingly, the operator can confirm the shift angle θ of the puncture needle 3 by visual observation using the shift angle image data, can match an orientation of the ultrasound probe 2 with the puncture needle 3 by moving the ultrasound probe 2, or can match an orientation of the puncture needle 3 with the ultrasound probe 2 by inserting the puncture needle 3 again.

The controller 11 judges whether or not the calculated shift angle θ of the puncture needle 3 is the predetermined threshold angle θ_{α} or more, and causes the output display 19 to output, if the shift angle θ of the puncture needle 3 is the threshold angle θ_{α} or more, warning information indicating that the shift angle θ of the puncture needle 3 is the threshold angle θ_{α} or more. Accordingly, if the shift angle θ of the puncture needle 3 is the threshold angle θ_{α} or more and is large, the controller 11 can warn the operator that the shift angle θ of the puncture needle 3 is the threshold angle θ_{α} or more and deal with the warning.

The controller 11 calculates the shift angle θ of the puncture needle 3 to the long axis direction (the scanning plane) of the ultrasound probe 2 viewed from the top. Accordingly, the operator can easily grasp the shift angle θ of the puncture needle 3 in the parallel method.

The controller 11 calculates the shift angle of the puncture needle to the short axis direction (a plane perpendicular to the scanning plane) of the ultrasound probe 2 viewed from the top. Accordingly, the operator can easily grasp the shift angle θ of the puncture needle 3 in the crossing method.

The controller 11 calculates the shift angle θ of the puncture needle 3 inserted by the parallel method using the one boundary position information of the extracted image of the puncture needle 3 in each of the regions and insertion reference position information of the puncture needle 3 (the central portion C1 in the short axis direction of the long axis end of the ultrasound probe 2). Accordingly, the shift angle θ of the puncture needle 3 in the parallel method can be calculated easily and with high accuracy.

The controller 11 calculates the shift angle of the puncture needle inserted by the crossing method using the plurality of boundary position information of the respective extracted images of the puncture needle 3 in the regions. Accordingly, the shift angle θ of the puncture needle 3 in the crossing method can be calculated easily and with high accuracy.

The image processor 16 makes the respective display colors as the representations of the extracted images of the puncture needle 3 in the regions different from one another for each of the regions, and synthesizes the respective ultrasound image data in the regions including the partial needle images which are made different in the display colors, to generate the composite ultrasound image data. The controller 11 displays the generated composite ultrasound image data on the output display 19. Accordingly, the operator can identify the images of the puncture needle 3 by visual observation for each of the regions, and can easily judge the position in the short axis direction of the puncture needle 3.

### (Modification)

A modification to the abovementioned embodiment will be described with reference to FIGS. 12A and 12B.

FIG. 12A is a histogram of a shift angle θ of a puncture needle 3. FIG. 12B is a diagram illustrating a history information image 70 of the shift angle.

In the modification, an ultrasound diagnostic apparatus U is used, like in the abovementioned embodiment, as an apparatus configuration. However, a nonvolatile memory such as an HDD in a controller 11 stores a puncture needle image display program in the modification instead of the puncture needle image display program in the abovementioned embodiment.

Then, an operation of the ultrasound diagnostic apparatus U will be described. The controller 11 performs puncture needle image display processing in the modification according to the puncture needle image display program in the modification stored in the nonvolatile memory, like in the abovementioned embodiment. The puncture needle image display processing in the modification is substantially similar to the puncture needle image display processing in the abovementioned embodiment, and different portions will be mainly described.

After starting to perform the puncture needle image display processing in the modification, the controller 11 first accepts input of identification information of an operator from the operator via an operation input unit 18. Steps S10 to S23 are executed. After step S23 is executed, the controller 11 stores a puncture method and the shift angle θ of the puncture needle 3 as history information of the shift angle of the puncture needle 3 in the nonvolatile memory in the controller 11 in association with the identification information first inputted only when the shift angle θ of the puncture needle 3 is first calculated while the puncture needle image display processing is performed once, for example. Steps S24 to S28 are executed. The larger the number of times the puncture needle image display processing in the modification is performed is, the more the history information of the shift angle of the puncture needle 3 for each operator stored in the nonvolatile memory in the controller 11 is.

The controller 11 is triggered by input of operator identification information to be displayed in the history information of the shift angle and an instruction to display the history information of the shift angle from the operator via the operation input unit 18, to read out the history information of the shift angle corresponding to the inputted identification information of the operator, generate display information for the read history information of the shift angle, and display the generated display information on an output display 19.

As the display information for the history information of the shift angle, a histogram in which a horizontal axis and a vertical axis respectively indicate the shift angle θ of the puncture needle 3 and a frequency can be displayed, as illustrated in FIG. 12A, for example. Not the histogram but a numerical value of the frequency for each shift angle θ of the puncture needle 3 may be displayed as the display information for the history information of the shift angle.

The display information for the history information of the shift angle may be set as a history information image 70 of the shift angle, as illustrated in FIG. 12B, for example. The history information image 70 of the shift angle is shift angle history information image in a parallel method, and includes an ultrasound probe image 71 and a puncture needle image 72. The ultrasound probe image 71 is a schematic plan view of a ultrasound probe 2 viewed from the top (from the side of a cable 5). The puncture needle image 72 is a plurality of arrow line images respectively representing insertion directions corresponding to accumulated shift angles 0 of the puncture needle 3 when the ultrasound probe image 71 is viewed from the top. An insertion position of the puncture needle image 72 is a central portion in a short axis direction of a long axis end of the ultrasound probe image 71.

As described above, according to the modification, the controller 11 stores the history information of the calculated shift angle θ of the puncture needle 3 in the nonvolatile memory in the controller 11. The controller 11 displays the stored history information of the shift angle of the puncture needle on the output display 19 as the histogram, the history information image 70, or the like. Accordingly, the operator can confirm the history information of the shift angle of the puncture needle 3 by visual observation, can recognize a habit and a tendency to insert the puncture needle, and improve a skill in inserting the puncture needle 3.

The description in the abovementioned embodiment is an example of an appropriate ultrasound diagnostic apparatus and a puncture needle shift angle calculation method according to the present invention, and the present invention is not limited to this.

Although the ultrasound diagnostic apparatus U is configured to generate and display the B mode image data as the ultrasound image data in the abovementioned embodiment, for example, the present invention is not limited to this. The ultrasound diagnostic apparatus U may be configured to generate and display tomographic image data in another mode as the ultrasound image data.

Although the ultrasound probe 2 in which the plurality of transducers in the long axis direction in three columns are arranged in the short axis direction has been described in the abovementioned embodiment, the present invention is not limited to this. The number of exclusive regions can also be increased by making the number of divisions (the number of transducers) in the short axis direction larger, for example, increasing the three columns to five columns, seven columns, . . . in the short axis direction or simultaneously using the plurality of transducers, for example.

Although the respective display colors as the representations of the partial images of the puncture needle 3 as the recognition object imaged in the independent transducers in the short axis direction are made different from one another in the embodiment and the modification, described above, the present invention is not limited to this. As the representations of the partial images of the puncture needle 3 imaged in the independent transducers in the short axis direction, other representations such as saturation, luminance, and flashing may be made different from one another.

A detailed configuration and a detailed operation of each of the units constituting the ultrasound diagnostic apparatus U according to the abovementioned embodiment can be appropriately changed without departing from the scope and spirit of the invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-075150, filed on Apr. 10, 2018, is incorporated herein by reference in its entirety.

What is claimed is:

1. An ultrasound diagnostic apparatus comprising:
a transmitter which transmits, to an ultrasound probe including a plurality of transducers which are arranged in a plurality of regions in a short axis direction and arranged in a long axis direction in each of the regions and can transmit and receive ultrasound independently in the plurality of regions in the short axis direction, a driving signal in each of the regions;
a receiver which receives a receiving signal in each of the regions from the ultrasound probe; and
a hardware processor which generates ultrasound image data from the received receiving signal in the region, extracts an image of a puncture needle inserted into a subject from the generated ultrasound image data in the region, and calculates a shift angle of the puncture needle to the ultrasound probe using boundary position information of the extracted image of the puncture needle in the region.

2. The ultrasound diagnostic apparatus according to claim 1, wherein the hardware processor displays shift angle display information representing the calculated shift angle of the puncture needle on a display.

3. The ultrasound diagnostic apparatus according to claim 1, wherein the hardware processor judges whether or not a shift angle corresponding to the calculated shift angle of the puncture needle is a predetermined threshold angle or more, and outputs, when the shift angle is the predetermined threshold angle or more, warning information indicating that the shift angle of the puncture needle is the predetermined threshold angle or more to an output unit.

4. The ultrasound diagnostic apparatus according to claim 1, wherein the hardware processor calculates the shift angle of the puncture needle to the long axis direction of the ultrasound probe viewed from the top.

5. The ultrasound diagnostic apparatus according to claim 1, wherein the hardware processor calculates the shift angle of the puncture needle to the short axis direction of the ultrasound probe viewed from the top.

6. The ultrasound diagnostic apparatus according to claim 1, wherein the hardware processor calculates the shift angle of the puncture needle inserted by a parallel method using boundary position information of the extracted image of the puncture needle in each of the regions and insertion reference position information of the puncture needle.

7. The ultrasound diagnostic apparatus according to claim 1, wherein the hardware processor calculates the shift angle of the puncture needle inserted by a crossing method using the plurality of boundary position information of the respective extracted images of the puncture needle in the regions.

8. The ultrasound diagnostic apparatus according to claim 1, wherein the hardware processor stores history information of the calculated shift angle of the puncture needle in a storage and displays the stored history information of the shift angle of the puncture needle on a display.

9. The ultrasound diagnostic apparatus according to claim 1, wherein the hardware processor makes respective representations of the extracted images of the puncture needle in the regions different from one another for each of the regions, synthesizes the respective ultrasound image data in the regions including the images of the puncture needle the representations of which are made different from one another to generate composite image data, and displays the generated composite image data on a display.

10. The ultrasound diagnostic apparatus according to claim 9, wherein the representation is at least one of a display color, saturation, luminance, and flashing.

11. A puncture needle shift angle calculation method comprising:
transmitting, to an ultrasound probe including a plurality of transducers which are arranged in a plurality of regions in a short axis direction and are arranged in a long axis direction in each of the regions and can transmit and receive ultrasound independently in the plurality of regions in the short axis direction, a driving signal in each of the regions;
receiving a receiving signal in each of the regions from the ultrasound probe;
generating ultrasound image data from the received receiving signal in each of the regions;
extracting an image of a puncture needle inserted into a subject from the generated ultrasound image data in the region; and
calculating a shift angle of the puncture needle to the ultrasound probe using boundary position information of the extracted image of the puncture needle in the region.
